(19) United States
(12) Patent Application Publication
(10) Pub. No.: US 2015/0141766 A1
(43) Pub. Date: May 21, 2015

(54) APPARATUS AND METHOD FOR OPTICAL MEASUREMENT OF CARDIOVASCULAR RECOVERY AND/OR REPIRATION RATE

(71) Applicant: FLFI-TFCH LTD., Rehovot (IL)

(72) Inventor: Ilya Fine, Rehovot (IL)

(21) Appl. No.: 14/503,395

(22) Filed: Sep. 30, 2014

Related U.S. Application Data
(60) Provisional application No. 61/884,975, filed on Sep. 30, 2013.
(60) Provisional application No. 61/884,202, filed on Sep. 30, 2013.

(51) Int. Cl. A61B 5/026; A61B 5/00; A61B 5/0205; A61B 5/029

(52) U.S. Cl. CPC A61B 5/0261; A61B 5/029; A61B 5/7278; A61B 5/7246; A61B 5/0205; A61B 5/746; A61B 5/7405; A61B 5/742; A61B 5/7225; A61B 5/7264; A61B 5/0816
USPC 600/301; 600/479

(57) ABSTRACT

The present disclosure relates to apparatus and method for optical measurement of cardiovascular recovery and/or a respiration rate. In some embodiments, an apnea detector generates an alert signal if the computed respiration rate drops below a threshold.

### BACKGROUND

In cardiovascular physiology, stroke volume (SV) is the volume of blood pumped from one ventricle of the heart with each beat. SV is calculated using measurements of ventricle volumes from an echocardiogram and subtracting the volume of the blood in the ventricle at the end of a beat (called end-systolic volume) from the volume of blood just prior to the beat (called end-diastolic volume). The term stroke volume can apply to each of the two ventricles of the heart, although it usually refers to the left ventricle. Stroke volume is an important determinant of cardiac output, which is the product of stroke volume and heart rate, and is also used to calculate ejection fraction, which is stroke volume divided by end-diastolic volume. Because stroke volume decreases in certain conditions and disease states, stroke volume itself correlates with cardiac function.

Ischemic heart disease (IHD) is a major health problem today. Patients are often not fully evaluated for cardiac function, and diastolic dysfunction of heart, which is often an earlier manifestation than systolic dysfunction, goes undetected. In heart failure commonly both systolic and diastolic dysfunction are seen. Systolic failure manifestations result from an inadequate cardiac output. Diastolic dysfunction manifestations relate to elevation of filling pressure. Early diagnosis and treatment is important in preventing irreversible structural alterations

Currently in the hospital a number of methodologies are used for the diagnostic of IHD. Among them tilt table tests, echocardiogram, electrophysiology tests, cardiac catheterization, X-Ray chest, heart MRI, CT and more.

a) The currently used home tests

Cardiovascular fitness represents the efficiency of the heart, lungs and vascular system in delivering oxygen to the working muscles so that prolonged physical work can be maintained. Many fitness and wellness programs aim to improve the cardiovascular strength and endurance. In order to measure cardiovascular strength of a subject the different stress tests are used. For example, the Bruce Protocol Treadmill test is used for evaluating cardiac fitness. The Bruce Protocol Treadmill Test is performed on a treadmill. As the Bruce Protocol Treadmill test is a maximal fitness test, one has to run continuously until get tired. The main disadvantage of these test is, that it requires using a treadmill equipment. The test is time and energy consuming and not very suitable for home for performing at daily basis. In addition, this test is based on subjective feeling of triteness and the related physiological parameters are not measured directly.
Another practical way to measure the improvement of the endurance or to make kind of a pre-diagnostic assessment is to measure the heart rate (HR) post stress recovery pattern. Post-exercise heart rate recovery, though a readily obtainable parameter and a powerful and independent predictor of cardiovascular and all-cause mortality in healthy adults and in those with cardiovascular diseases, is often overlooked as an indicator of cardiovascular fitness. Heart rate recovery (HRR) is thought to be major characteristics of parasympathetic reactivation but not providing comprehensive information regarding the endurance improvement. So the heart rate behavior only can't provide comprehensive information about the cardiovascular ability to supply the blood but rather indirect indications via the regulatory mechanics. Maximal heart rate is not an appropriate indicator of the amount of blood being pumped around the body, especially not for trained endurance athletes.
All tasks related to the heart rate are not addressing the heart pump systolic and diastolic performance during the cycle of heart beat. Together with the SV provides a direct indication of the heart performance. If SV increases with endurance training, it means that more blood is pumped around the body with every heart bit beat. Therefore, a reduction in maximal heart rate does not result in the body receiving less blood. In fact, the opposite is true, as the reduced heart rate is more than compensated for by higher stroke volume. The reason for increased stroke volume is an increase in the end diastolic volume, the volume of blood in the left ventricle just before contraction. The major difference in the endurance trained heart is a bigger stroke volume. The trained heart gets bigger and pumps more blood each.

b) Parameters of cardiac functioning

Training results in an increase in stroke volume (SV) in maximal cardiac and output (CO) which is defined as the product of SV and heart beats per minute.

### C) Respiration Rate (RR).

One of the major factors which might influence left ventricular stroke volume in normal subjects during the respiratory cycle is the variation in filling time of the ventricle. Stroke volume variation is a naturally occurring phenomenon in which the arterial pulse pressure falls during inspiration and rises during expiration due to changes in intra-thoracic pressure secondary to negative pressure ventilation (spontaneously breathing). Therefore an information about the stroke volume can be used to extract the respiration rate.

Currently there is no practical home based method to estimate changes in CO and related parameters during the cardiac cycles The current invention proposes a new methodology for non-invasive measurement of the hemorheological parameters at peripheral locations on the body and are related to CO. The non-invasive assessment of these parameters can be used for fitness, wellness and medical applications

### SUMMARY

Embodiments of the invention relate to methods and apparatus of optically and non-invasively (i) measuring a cardiovascular recovery metric or a respiration rate and (ii) detecting certain events related to respiration rate or a change therein. In particular, it is possible to measure a blood-shear parameter and to derive the cardiovascular recovery metric or reparation rate therefrom.

Some embodiments relate to a cardiovascular recovery metric. When a subject exercise or subjects his/her cardiovascular system to an elevated load, his/her pulse rate increases and his/her stroke volume parameter increases. After the exercise ceases, or decreases in intensity, his/her cardiovascular system no longer has the same need to oxygenate the body as was previously required during more intense activity. As a result, the cardiovascular system returns to a lower rate of activity. However, the amount of time required for this to occur differs between subjects. More fit subjects tend to have a significantly lower recovery time than those subjects who are not in shape (e.g. overweight, unaccustomed to exercise, etc).

Knowledge of the cardiovascular recovery time may be useful in diagnosing or prognosticating heart disease, and there is a need to encourage people to measure this physiological parameter. Unfortunately, it may require a certain amount of time to accurately measure this ‘recovery time’ (for example, minutes) and many subjects are unlikely to comply—instead, this parameter may just go unmeasured.

Embodiments of the invention relate to an apparatus and method for accurately, quickly, optically and non-invasively measuring the cardiovascular recovery time.

In particular, it is now disclosed that the correlation between a per-cycle stroke-volume and the per-cycle pulse rate may be measured (e.g. using any apparatus disclosed herein), and the cardiovascular recovery metric may be computed therefrom. This topic will be further discussed below with reference to FIG. 5A.

Some embodiments relate to respiration rate. In particular, it is now disclosed an apparatus and method which (i) monitor cardiac-cycle specific stroke volume parameters to compute a stroke volume parameter signal; and (ii) subject this stroke volume parameter signal to a temporal analysis. The respiration rate may be derived from the temporal analysis—e.g. by computing a dominant frequency.

### BRIEF DESCRIPTION OF THE DRAWINGS

FIG. 1A is an illustration of a DLS measurement based system

FIG. 1B is an illustration of a DLS measurement based system for measuring one or more blood parameters.

FIG. 2-3 are flow charts of a technique for computing a cardiac-cycle specific stroke volume parameter of a subject.

FIG. 4 illustrates signal form characteristics.

FIGS. 5A-5B relate to correlating between the per-cycle stroke volume parameter and the per-cardiac-cycle pulse rate.

FIGS. 6A-6B relate to a second application of the technique for deriving the per-cycle stroke volume parameter.

FIG. 6C is a flow chart of a method for generating an alert signal according to detection of classifier events.

FIG. 7 is an illustration of a DLS measurement based system for measuring one or more blood parameters.

FIG. 8 is an illustration of a system for generating an analog electrical signal.

FIG. 9 is a circuit diagram of an exemplary analog electronic assembly.

FIGS. 10-11 illustrate the detector-generated electrical signal or ‘analog substraction-derivatives’ thereof.

FIGS. 12A-12B illustrate identifying signal form parameters of a cardiac cycle.

FIG. 13A-13B illustrate computing a corretlation between the multi-cycle stroke volume parameter data set and cycle-specific heart rate/pulse data set.

FIG. 14 graphs a correlation between respiration rate parameters.

FIG. 15 is a block diagram of a system including a source of light, a photodetector assembly, electronic circuitry and a data-presentation device.

FIGS. 16-18 and 19A-19B are flow charts of techniques for measuring physiological or blood parameter(s).

### DESCRIPTION OF EMBODIMENTS

Some embodiments of the present invention relate to methods and apparatus that were disclosed in U.S. 61/884,202 and U.S. 61/884,975 which were both filed on Sep. 30, 2013 and which are both incorporated herein by reference in its entirety. In some embodiments, any feature or combination of features described in the present document may be combined with any feature of combination of features described in applications U.S. 61/884,202 and/or U.S. 61/884,975.

The claims below will be better understood by referring to the present detailed description of example embodiments with reference to the figures. The description, embodiments and figures are not to be taken as limiting the scope of the claims. It should be understood that not every feature of the presently disclosed methods and apparatuses for handling error correction is necessary in every implementation. It should also be understood that throughout this disclosure, where a process or method is shown or described, the steps of the method may be performed in any order or simultaneously, unless it is clear from the context that one step depends on another being performed first. As used throughout this application, the word “may” is used in a permissive sense (i.e., meaning “having the potential to’), rather than the mandatory sense (i.e. meaning “must”).

WO 2008/053474, incorporated herein by reference in its entirety, discloses a system and method for in vivo measurement of biological parameters.

In particular, WO 2008/053474 discloses a novel optical technique suitable for the in vivo measurement in a subject utilizing dynamic light scattering (DLS) approach. The effect of DLS are utilized for the measurement of variety of blood related parameters, such as viscosity of the blood and blood plasma, blood flow, arterial blood pressure and other blood chemistry and rheology related parameters such as concentration of analyte (e.g. glucose, hemoglobin, etc.), oxygen saturation etc.

DLS is a well-established technique to provide data on the size and shape of particles from temporal speckle analysis. When a coherent light beam (laser beam, for example) is incident on a scattering (rough) surface, a time-dependent fluctuation in the scattering property of the surface and thus in the scattering intensity (transmission and/or reflection) from the surface is observed. These fluctuations are due to the fact that the particles are undergoing Brownian or regular flow motion and so the distance between the particles is constantly changing with time. This scattered light then undergoes either constructive or destructive interference by the surrounding particles and within this intensity fluctuation information is contained about the time scale of movement of the particles. The scattered light is in the form of speckles pattern, being detected in the far diffraction zone. The laser speckle is an interference pattern produced by the light reflected or scattered from different parts of an illuminated surface. When an area is illuminated by laser light and is imaged onto a camera, a granular or speckle pattern is produced. If the scattered particles are moving, a time-varying speckle pattern is generated at each pixel in the image. The intensity variations of this pattern contain information about the scattered particles. The detected signal is amplified and digitized for further analysis by using the autocorrelation function (ACF) technique. The technique is applicable either by heterodyne or by a homodyne DLS setup.

The kinetics of optical manifestations of two kinds of physiological signals is measured in vivo: the pulsatile signal associated with heart beats and the post-occlusion optical signal which is induced by an artificially generated blood flow cessation. The light transmission and/or reflection signals are used as a control of the physiological response. This kind of control measurement can be carried out simultaneously with the DLS reflection measurement. The mutual correspondence between DLS and standard optical signals is subject to a comparison analysis.

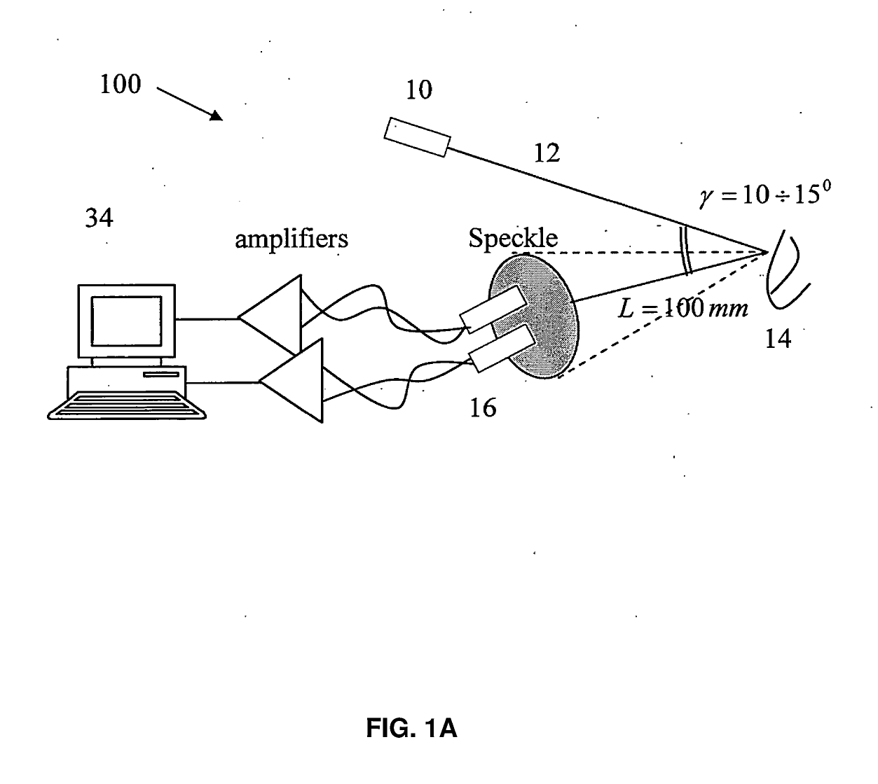

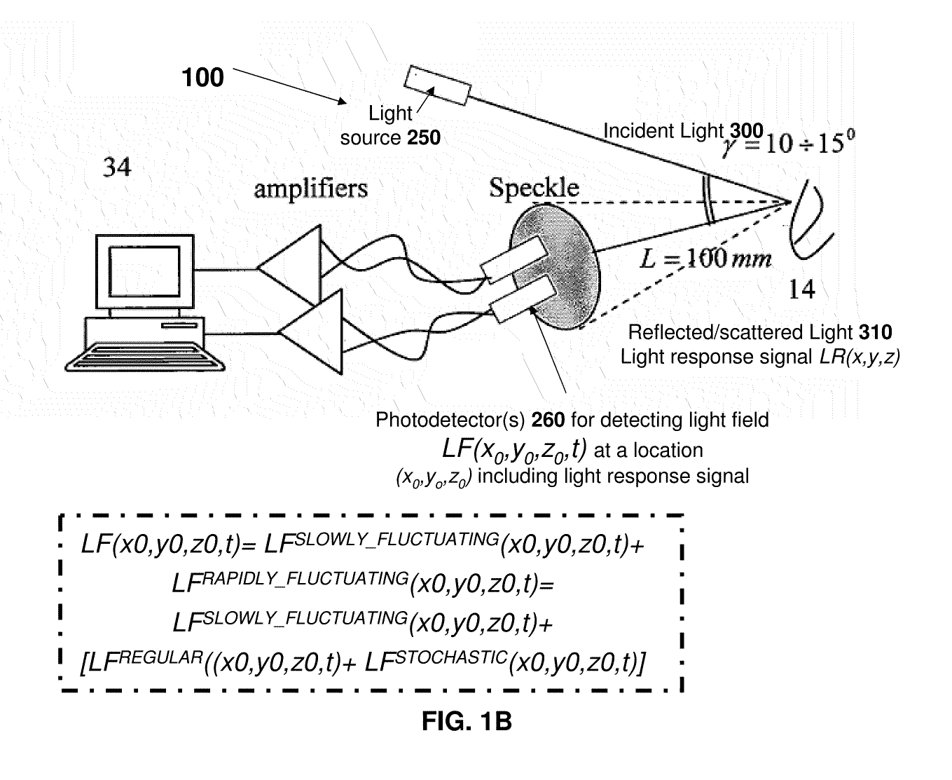

FIG. 1A is taken from WO 2008/053474, incorporated by reference in its entirety.

PCT/US2010/056282, incorporated by reference in its entirety, discloses a specific system whereby first and second photodetectors respectively generate first and second analog signals, from which a digital analog signal may be computed. FIG. 1B is reproduced from PCT/US2010/056282.

Embodiments of the present invention relate to the use of systems disclosed in WO 2008/053474 and PCT/US2010/056282 to optically and non-invasively detect two types of physiological parameters that, in the prior art, could not be detected optically and non-invasively.

### Definitions

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

Analog electrical signals or light fields may comprises more than one sub-signal added together in a single electrical (or optical) signal. For example, an analog electrical signal derived from a light field detected by a photodetector that (i.e. where scattered light that is scattered from particles within a fluid contributed to the light field) may be the sum of: (i) a first component (i.e. analog electrical sub-signal) attributable to ambient light (e.g. sunlight); (ii) a second component attributable to skin light-modulating effects; (iii) a third component attributable to regular fluctuations in light intensity due to the presence of a fluorescent bulb and (iv) a fourth component attributable to scattered light that is scattered from particles within a fluid contributed to the light field. Each component or sub-signal of the analog electrical signal is associated with a different respective amount of power.

In some examples, for an analog signal generated by a photodetector, the relative power contribution to overall analog signal power attributable to ambient light is relatively high (i.e. the first component), while the relative power contribution to overall analog signal power attributable to scattered light that is scattered from particles within a fluid is relatively low (i.e. second component).

In general, both a signal and a sub-signal have power levels—the fraction of the power level of the overall signal attributable to a particular portion of the signal or sub-signal is the ‘power fraction’ of the sub-signal or signal component. In the example of the previous paragraph, the power fraction of the overall analog electrical signal due to the ambient light component may be significant (e.g at least 0.1 or at least 0.3 or at least 0.5) while the power fraction of the overall analog electrical signal due to the ‘light scattering’ component (i.e. fourth component) may be relatively low—for example, at most 0.1 or at most 0.05 or at most 0.01).

Embodiments of the present invention relate to generating a ‘hybrid’ signal. A ‘hybrid signal’ derived from a plurality of input analog signals is any non-zero or non-trivial mathematical combination of the input analog signals—i.e., including multiplication, addition, subtraction, etc. The term ‘hybrid’ refers to the fact that the output (or hybrid) signal relates to more than one input signal, and is not restricted to a single input.

Embodiments of the present invention relate to photodetectors (any technology may be used including those listed herein or any other technology). In some embodiments, each photodetector is not infinitesimally small but rather has a size. The ‘distance’ between photodetectors relates to a centroid-centroid distance.

In some embodiments, a light field is comprised of more than on component. Whenever light is generated and reflected or scattered (or modulated in any other manner) to introduce photons into (or to pass through) a certain location (and/or to illuminate the location), this light ‘contributes to’ or ‘influences’ the local light field at that certain location.

Embodiments of the present invention relate to optically measuring a paremter relating to a subject. In different embodiments, this subject is human, or a mammal other than human, or to a warm-blooded animal other than mammals (e.g. birds).

Whenever a power level of a second signal is ‘significantly less’ than a power level of a first signal, a ratio between a power level of the second signal and a power level of the first signal is at most 0.5 or at most 0.3 or at most 0.2 or at most 0.1 or at most 0.05 or at most 0.01.

Some embodiments of the present invention are described for the specific case of only two photodetectors and/or measuring a light field in two locations. The skilled artisan will appreciate that this is not a limitation, any teaching disclosed herein may relate to the case of more than two photodetectors or detecting light fields in more than two locations. Thus, two photodetectors refers to ‘at least two,’ ‘two locations’ refers to at least two, and so on.

A Discussion of FIG. 1B

FIG. 1B, reproduced from PCT/US2010/056282, is an illustration of a DLS measurement based system for measuring one or more blood parameters. System 100 includes a light source unit 250 (e.g. laser) for generating at least partially coherent light; optical arrangement (not shown) including focusing optics and possibly also collecting optics; and a detection unit including a photodetector 260. A focused beam of light 300 produced by laser 250 (e.g., a semiconductor laser) is used as a localized light source. In a non-limiting example, a light source unit 250 may be a laser diode (650 nm, 5 mW) or VCSEL (vertical cavity surface emitting laser). The light response i.e. the reflected and/or transmitted light returned from the localized region of the subject's surface 14 (subject's finger in the present example) illuminated with the localized light source 250, can be collected in a determined distance L (in a non-limiting example, L=100 mm) either directly by a detector or via multimode fiber optics. In a non-limiting example, the multimode fiber optics may be a bi-furcated randomized optical fiber where one optical entrance is connected to the detector and another one is optically coupled with the laser diode. In particular, as shown in FIG. 1, system 100 includes at least one laser diode and at least one photodetector (for example, photodiode(s)) appropriately positioned in the reflection-mode measurement set-up.

The photodetector 260 is positioned in space at location (x0,y0,z0) and is configured to detect a light field LF(x₀, y₀, z₀)—i.e. the light field that exists/prevails at point (x₀, y₀, z₀). Typically, the light detected by photodetector 260 comes from a number of sources including but not limited to (A) reflected light 310 which is reflected from and/or scattered by the biological tissue; and (ii) ambient light. Thus, it is possible to write:

LF(x₀, y₀, z₀)=LF_{reflected}(x₀, y₀, z₀)+LF_{ambient}(x₀, y₀,z₀)+other term(s) (EQ. 1)

Throughout the present disclosure, LF denotes a light field.

When light from light source 250 is incident upon biological tissue, (i) a first portion of the incident light is reflected from or scattered from “Brownian particles” (i.e. particles undergoing Brownian motion within a liquid—for example, red blood cells or thromobcytes) to generate a first light response signal whose magnitude/intensity varies stochastically and rapidly in time—this first light response signal is referred to as LF_{reflected}_{_{—}}_{brownian} (x₀, y₀, z₀); (ii) a second portion of the incident light is reflected from stationary biological matter other than Brownian particles—for example, from skin cells, etc—this second portion of the incident light generates a second light response signal whose magnitude/intensity varies at most “slowly” and/or is not stochastic in time—this second light response signal is referred to as LF_{reflected}_{_{—}}ₙₒₙ_{_{—}}_{brownian}(x₀, y₀, z₀);

Thus, it is possible to write:

LF_{reflected}(x₀, y₀, z₀)=LF_{reflected}_{_{—}}ₙₒₙ_{_{—}}_{brownian}(x₀, y₀, z₀)+LF_{reflected}_{_{—}}_{brownian}(x₀, y₀, z₀)+other term(s) (EQ. 2)

In some embodiments, LF_{reflected}_{_{—}}_{brownian}(x₀, y₀, z₀) is indicative of a dynamic light scattering parameter. Unfortunately, in many clinical situations

$$\frac{{LF}_{reflected \_ brownian} ( {x}_{0} , {y}_{0} , {z}_{0} )}{LF ( {x}_{0} , {y}_{0} , {z}_{0} )} and \text{/} or$$

$$\frac{{LF}_{reflected \_ brownian} ( {x}_{0} , {y}_{0} , {z}_{0} )}{{LF}_{reflected \_ n o n \_ brownian} ( {x}_{0} , {y}_{0} , {z}_{0} )} and \text{/} or$$

$$\frac{{LF}_{reflected \_ brownian} ( {x}_{0} , {y}_{0} , {z}_{0} )}{{LF}_{reflected \_ ambient} ( {x}_{0} , {y}_{0} , {z}_{0} )}$$

is “small” (for example, less than 0.1 or less than 0.01 or even smaller).
Embodiments of the present invention relate to apparatus and methods for “boosting” the relative contribution to an analog electrical signal of a component indicative of a dynamic light scattering measurement—for example, boosting the relative contribution of an analog electrical signal indicative of LF_{reflected}_{_{—}}_{brownian}(x₀, y₀, z₀).

It is noted that, typically, LF_{ambient}(x₀, y₀, z₀) (see Eqn. 1) and LF_{reflected}_{_{—}}ₙₒₙ_{_{—}}_{brownian}(x₀, y₀, z₀) (see Eqn. 2) have an intensity that is either: (i) “slowly” fluctuating (for example, substantially constant or fluctuating at a rate less than 50 HZ); and/or (ii) “regularly behaved” and non-stochastic. One example of a “rapidly” fluctuating light signal that is regularly behaved and non-stochastic is light from a fluorescent light bulb operating at 50 HZ or 60 HZ. In contrast, the intensity of “speckles pattern light signal” LF_{reflected}_{_{—}}_{brownian}(x₀, y₀, z₀) varies stochastically and rapidly—i.e. at a rate that is at least 50 HZ or at least 100 HZ or at least 200 HZ, depending on diffusion coefficient of the Brownian particle.

Thus, it is possible to write:

$$LF ( {x}_{0} , {y}_{0} , {z}_{0} ) = {LF}_{slowl y \_ fluctuating} ( {x}_{0} , {y}_{0} , {z}_{0} ) + \underbrace{[ {LF}_{regular} ( {x}_{0} , {y}_{0} , {z}_{0} ) + {LF}_{stochastic} ( {x}_{0} , {y}_{0} , {z}_{0} ) ]}_{rapidly - fluctuating} + other terms \qquad ( EQ . 3 )$$

where (i) LF_{slowly}_{_{—}}_{fluctuating}(x₀, y₀, z₀) is due to ambient light LF_{ambient}(x₀, y₀, z₀) and/or light reflected from biological tissue other than Brownian particles LF_{reflected}_{_{—}}ₙₒₙ_{_{—}}_{brownian(x}₀, y₀, z₀); (ii) rapidly-fluctuating (i.e. at a rate of greater than 50 HZ and/or 100 HZ and/or 200 HZ) LF_{regular}(x₀ , y₀, z₀) is due to ambient light LF_{ambient}(x₀, y₀, z₀); and LF_{stochastic}(x₀, y₀, z₀)=LF_{reflected}_{—}_{ₙₒₙ}_{brownian}(x₀, y₀, z₀)/

For the present disclosure, “slowly fluctuating” refers to fluctuations at a rate of less than 50 HZ, while “rapidly fluctuating” refers to regular or stochastic fluctuations at a rate that exceeds 50 HZ (for example, at least 100 HZ or at least 200 HZ).

It is noted that: (i) LF_{stochastic}(x₀, y₀, z₀) is the portion of LF(x₀, y₀, z₀) that may be subjected to DLS analysis to yield one or more blood-related parameters; and (ii) in most clinical situations,

$$\frac{{LF}_{stochastic} ( {x}_{0} , {y}_{0} , {z}_{0} )}{LF ( {x}_{0} , {y}_{0} , {z}_{0} )}$$

is relatively “small” (for example, less than 0.1 or less than 0.01 or even smaller).

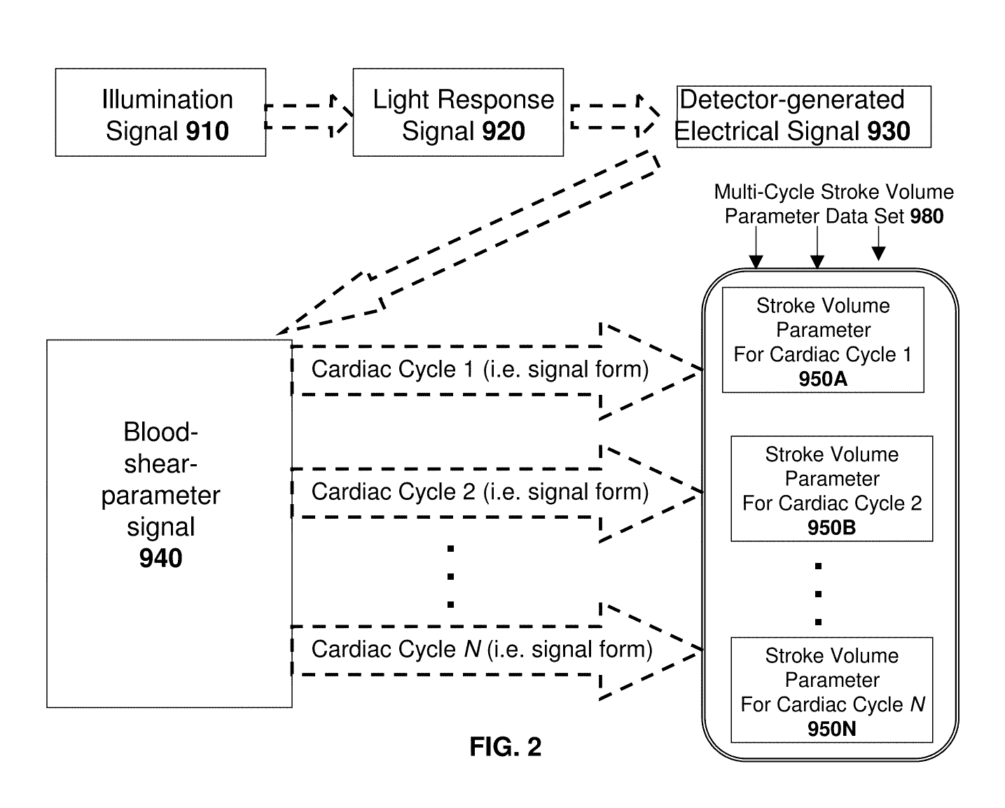

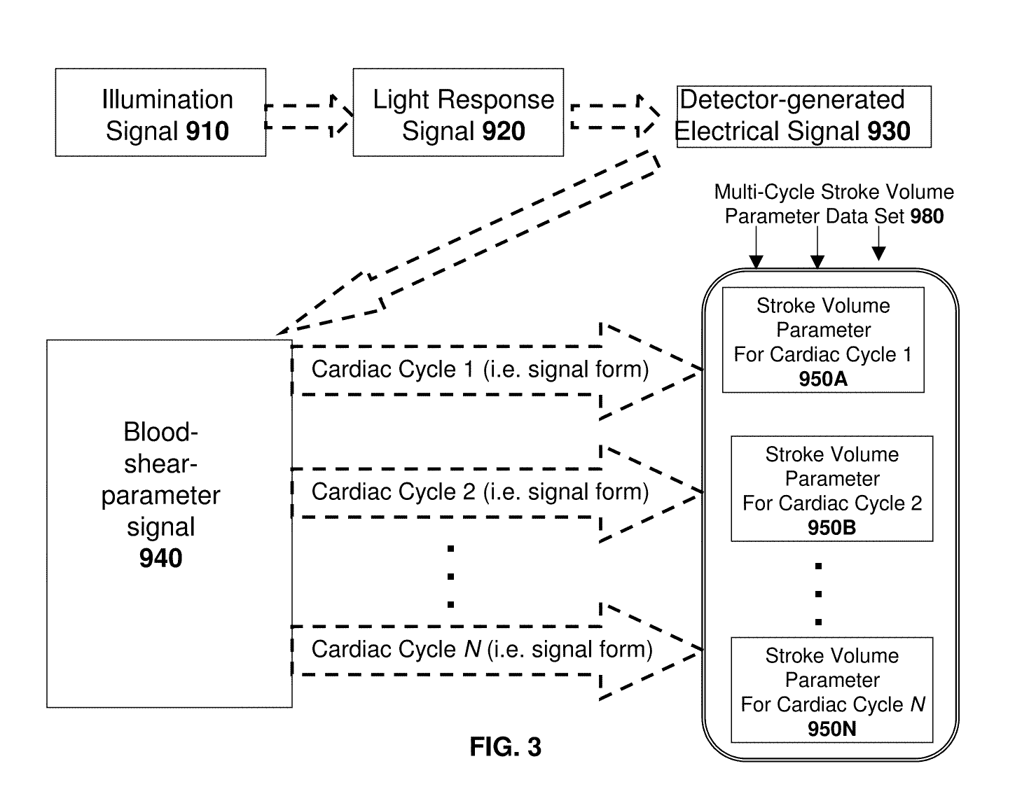

A Discussion of FIG. 2

FIG. 2 is a flow chart of a technique for computing a cardiac-cycle specific stroke volume parameter of a subject.

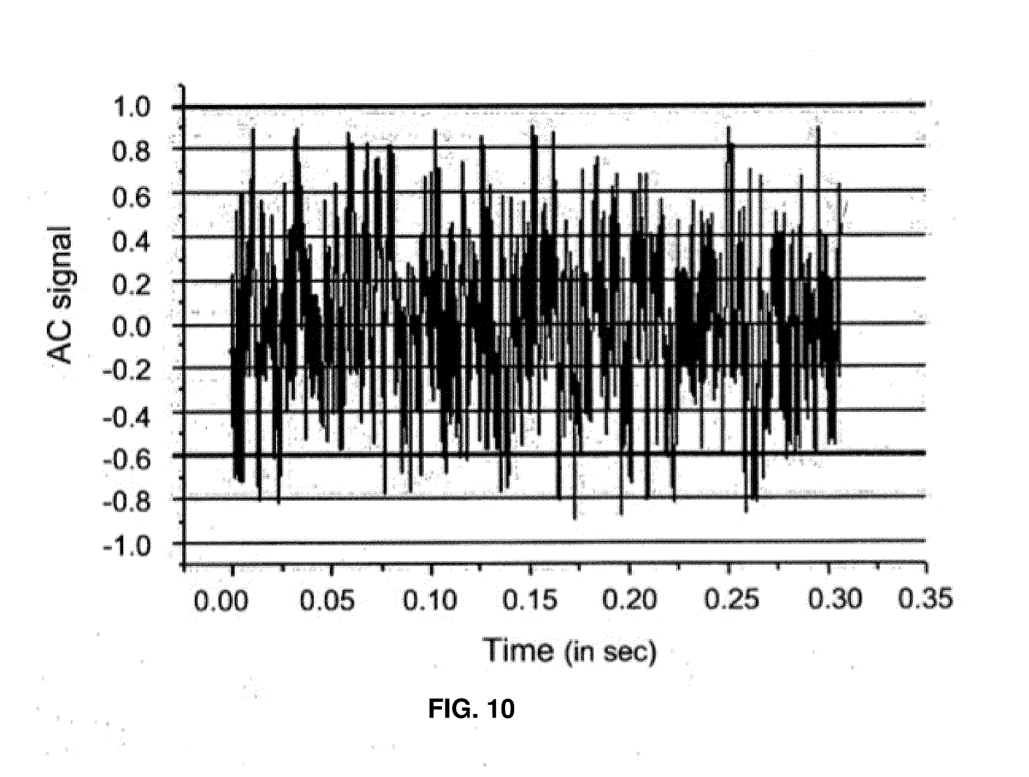

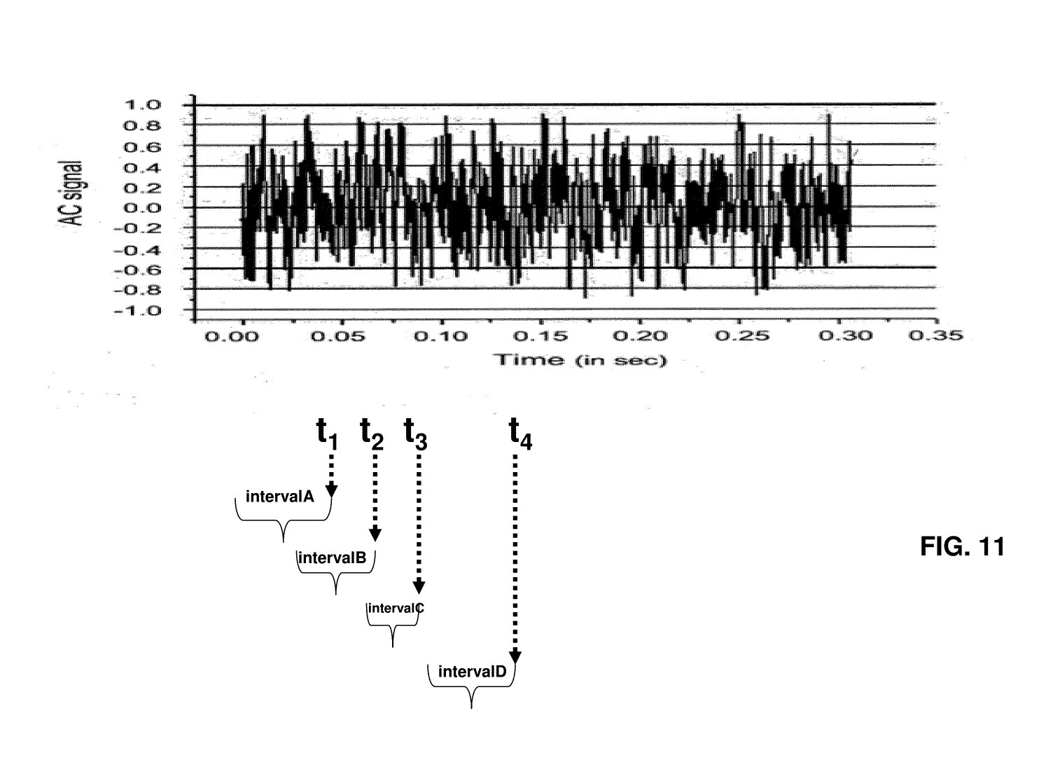

An illumination signal (e.g. from element 10 of FIG. 1A) induces a light response signal 920 (or light field) by reflection and/or transmission and/or deflection by biological tissue. This light reponse signal is detected by photodectors (e.g. first and second photodetectors) to generate an electrical signal 930 descriptive of light scattering (see FIGS. 10-11). Optionally but preferably, an analog difference signal (e.g. PCT/US2010/056282) is computed.

The analog signal, difference signal or a deritative thereof (e.g. digitized) is temporally analyzed to compute a blood-shear-parameter signal 940. The time scale of blood-shear-parameter signal 940 (i.e. related to shear—e.g. a rheological parameter which varies in time) is typically much large than the time scale of electrical signal generated by the photodetector which fluctuates very rapidly in time.

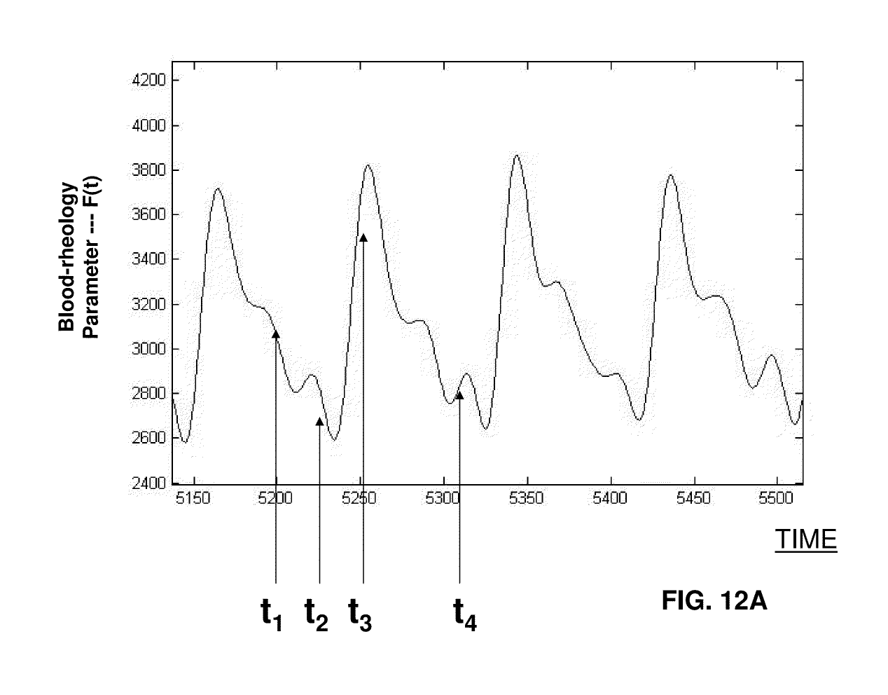

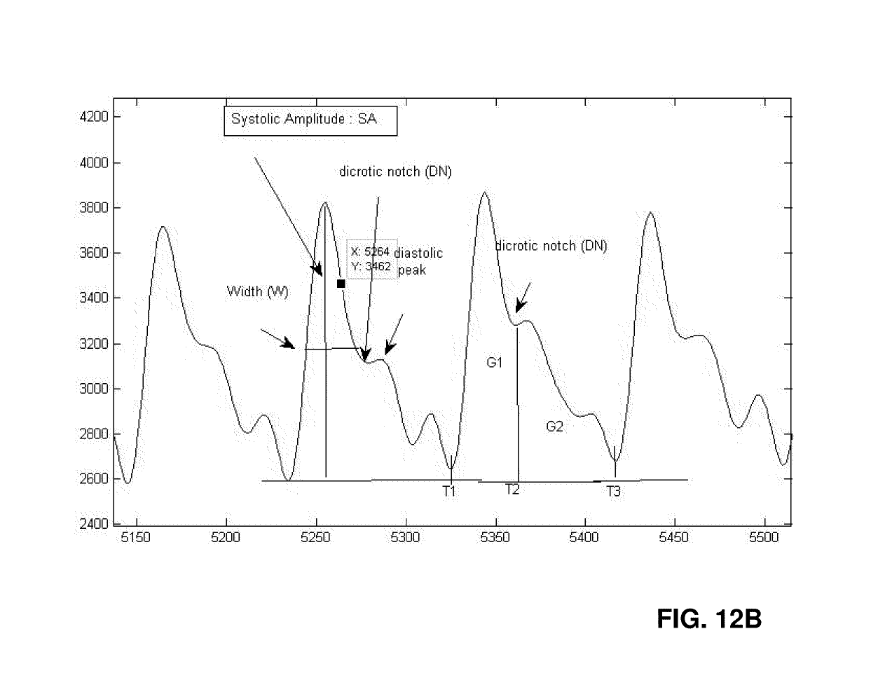

As illustrated in FIG. 2, even though this blood-shear-parameter signal fluctuates in time at a much slower rate, its value does, in fact, vary in time, and temporal patterns of this signal may be analyzed. For example, for a plurality of cardiac cycles, signal form parameters (i.e. see FIGS. 12A-12B) may be determined in any manner. According to the signal form parameters, it is possible to compute a stroke volume parameter per cycle—e.g. as disclosed in U.S. 61/884,202 and/or U.S. 61/884,975.

Collectively, these stroke volume parameters per volume comprise a data set, labeled as 980 of FIG. 2.

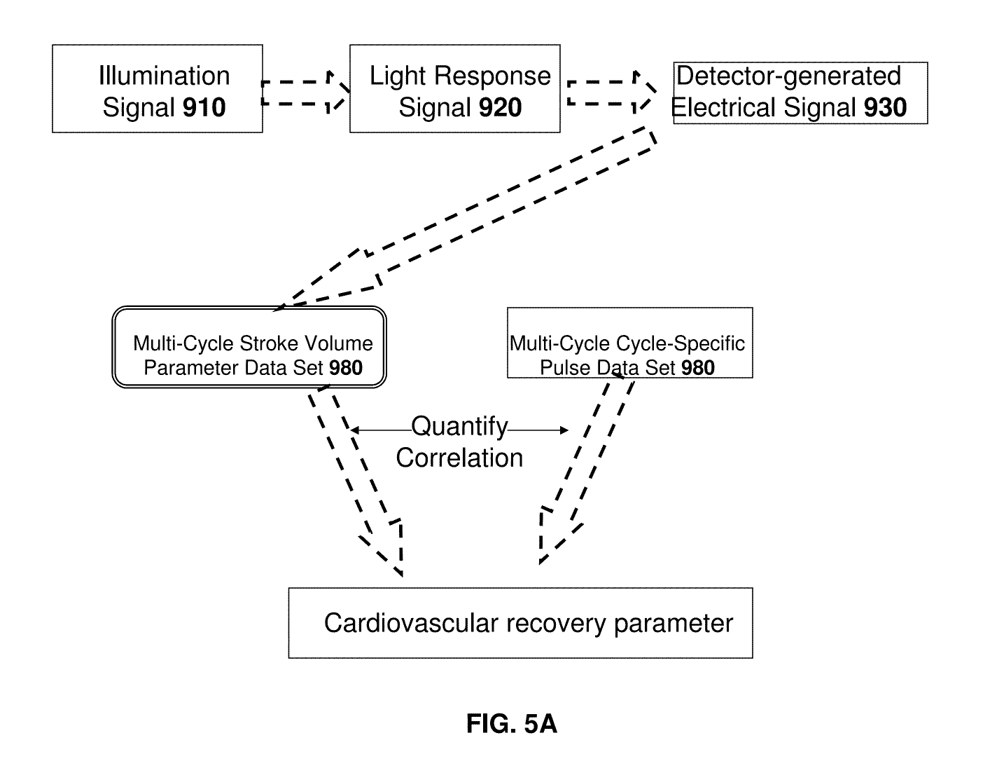

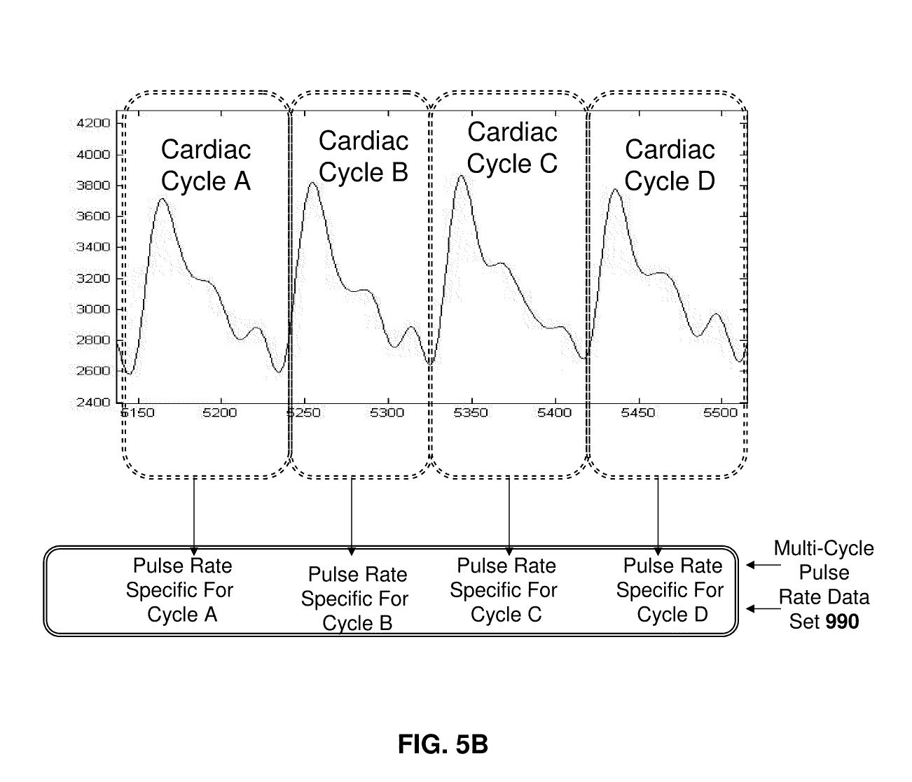

This data set may be analyzed to either (i) compute the cardiovascular recovery parameter (FIG. 5A) or (ii) compute the respiratory rate.

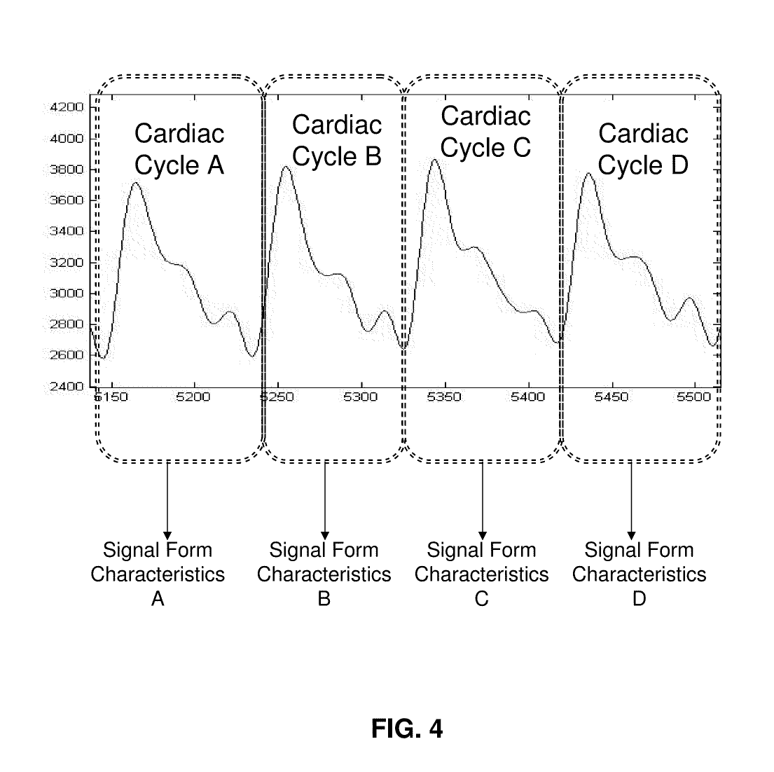

FIG. 4 illustrate ssignal form characteristics—i.e. each cardiac cycle has it's own signal form characteristic—e.g. time of beginning of the cycle, time of end of the cycle, diochroic notch-related times, etc—the skilled artisan is referred to U.S. 61/884,202 and/or U.S. 61/884,975. It is possible to integrate the blood-shear-parameter over time intervals bounded by times of the signal form of the cardiac cycle.

FIGS. 5A-5B relate to correlating between the per-cycle stroke volume parameter and the per-cardiac-cycle pulse rate. As shown in FIG. 5B, the pulse rate is not constant but may fluctuate in time—for each cardiac cycle, it is possible to compute a representative pulse rate.

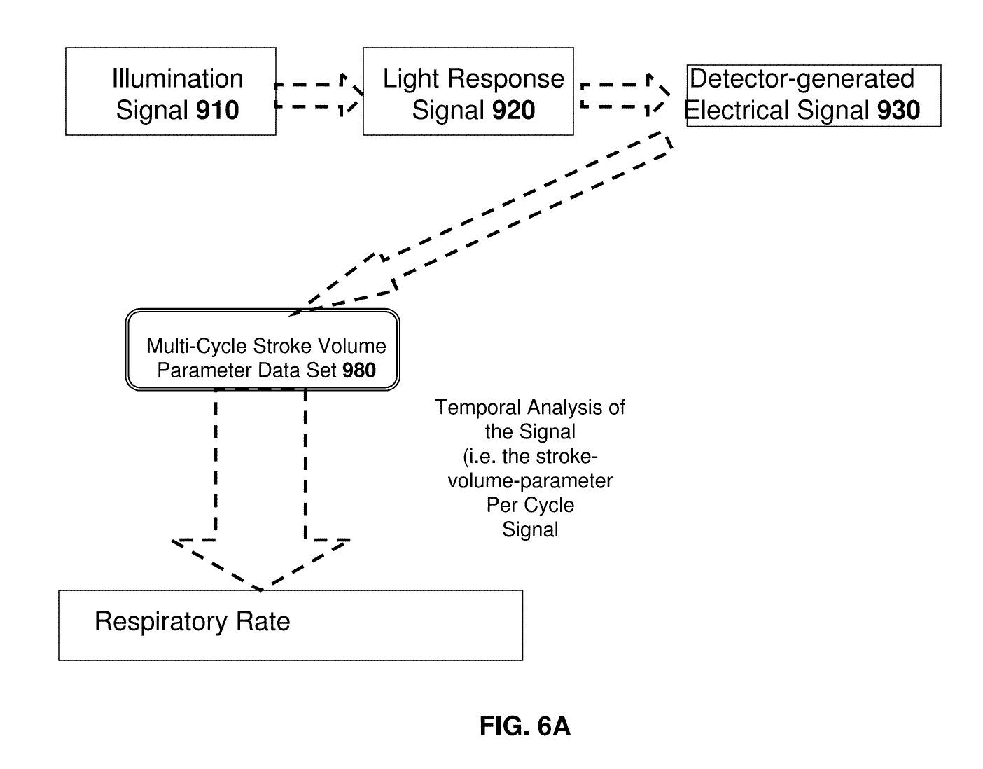

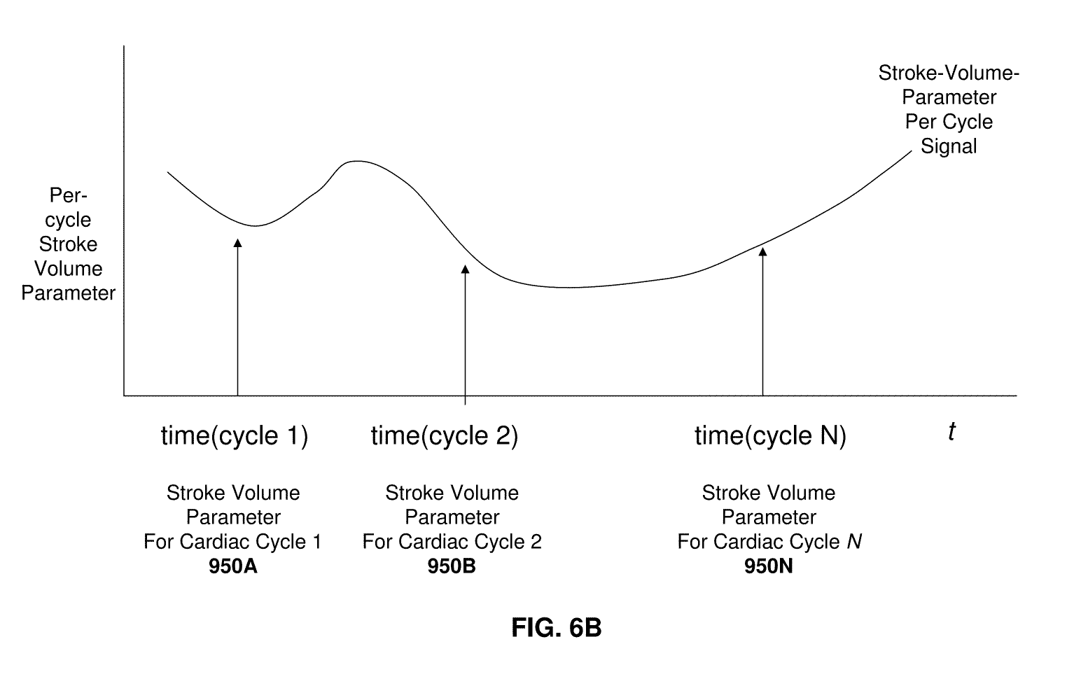

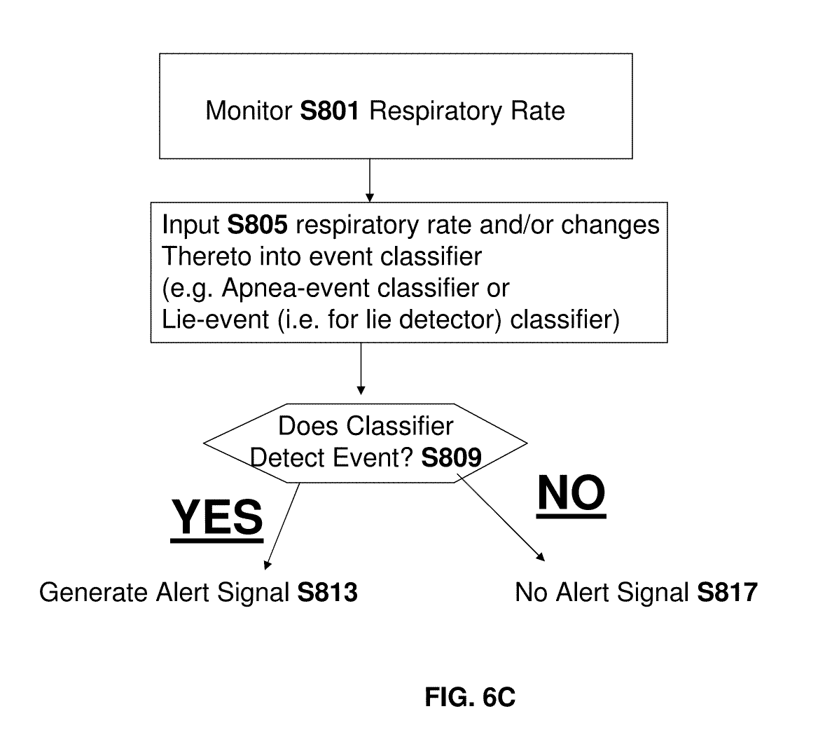

FIGS. 6A-6B relate to a second application of the technique for deriving the per-cycle stroke volume parameter. A respiratory rate may be computed, for example, by analyzing temporal patterns of the signal associated with the per-cycle stroke volume parameter. For example, if the dominant frequency of this per-cycle-stroke-volume parameter is relatively high, this indicates a relatively high respiratory rate. If the dominant frequency of this per-cycle-stroke-volume parameter is relatively low, this indicates a relatively low respiratory rate

In FIG. 6C, this may be employed, for example to detect apnea or lying or any other event associated with respiratory rate. For example, if the subject's pulse drops below a certain threshold (i.e. apnea event) (e.g. for a certain period of time), an alarm signal is generated.

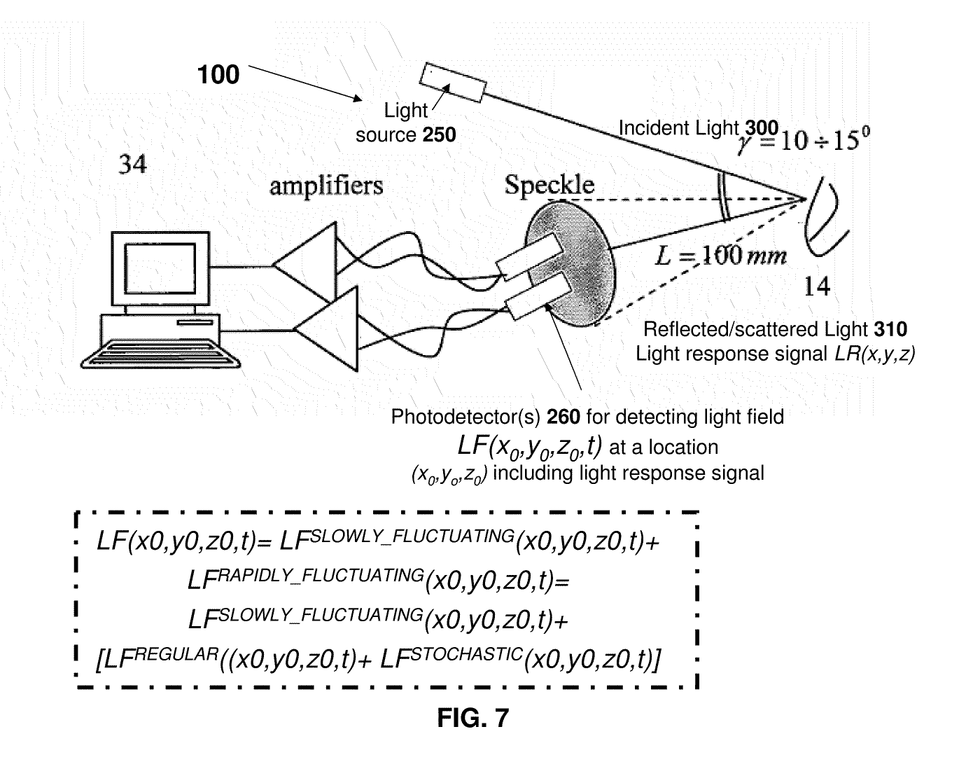

FIG. 7 is identical to FIG. 1B.

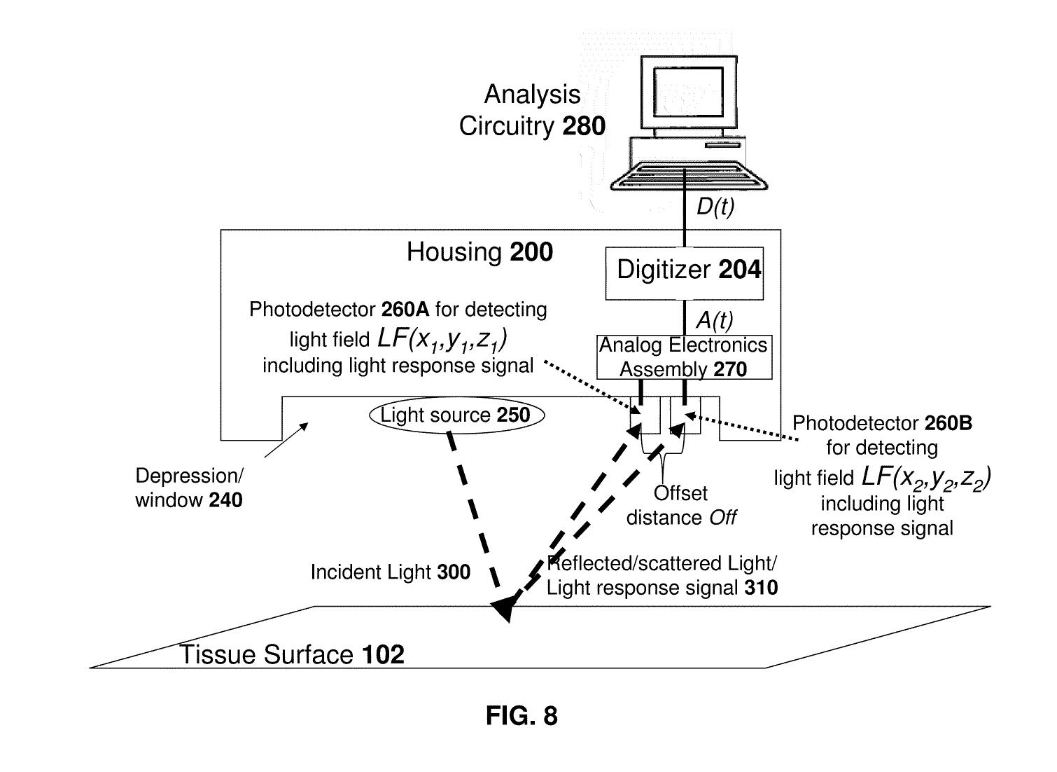

FIG. 8 is an illustration of a system for generating an analog electrical signal A(t) that includes a relatively “large” component (for example, at least 0.01 or at least 0.1 or at least 0.2 or least 0.3 or at least 0.5 or least 0.8) that is indicative of a time-varying “speckles pattern light signal” received by one or more photo-detectors. This analog signal may be converted, using A to D converter or digitizer 204, into a digital signal D(t). The digital signal may be subjected to any analysis described in WO 2008/053474 by digital circuitry 280 to determine one or more blood parameters—for example, temporal autocorrelation or power spectrum techniques.

In a non-limiting example, the data is collected at 22 KHz sampling rate and 16-bit resolution, and then analyzed by digital circuitry 280.

In the system of FIG. 8, light is received and detected by a plurality of photodetectors including: (i) photodetector 260A for detecting the light field LF(x₁, y₁, z₁) at location (x₁, y₁, z₁); (ii) photodetector 260B for detecting the light field LF(x₂, y₂, z₂) at location (x₂, y₂, z₂). Photodetector 260A generates a first analog electrical signal A₁(t) from LF(x₁, y₁, z₁). Photodetector 260B generates a second analog electrical signal A₂(t) from LF(x₂, y₂, z₂). Analog electronics assembly 270 receives the first A₁(t) and second A₂(t) analog electrical signals, and generates a “difference” between these two signals A₁−A₂(t) to produce analog electrical signal A(t) which is digitized. Photodetectors 260B and 260B are positioned so that: (i) they are close enough together so that LF_{ambient}(x₁, y₁, z₁)≈LF_{ambient}(x₂, y₂, z₂),

LF_{reflected}_{_{—}}ₙₒₙ_{_{—}}_{brownian}(x₁, y₁, z₁)≈LF_{reflected}_{_{—}}ₙₒₙ_{_{—}}_{brownian}(x₂, y₂, z₂),
LF_{slowly}_{_{—}}_{fluctuating}(x₁, y₁, z₁)≈LF_{slowly}_{_{—}}_{fluctuating}(x₂, y₂, z₂) and
LF_{regular}(x₁, y₁, z₁)≈LF_{regular(x}₂, y₂, z₂); and (ii) they are far enough from each other such that the rapidly fluctuating LF_{stochastic(x}₁, y₁, z₁) and LF_{stochastic}(x₂, y₂, z₂) are not correlated with each other.

There is no limitation on any separation distance between (x₁, y₁, z₁) and (x₂, y₂, z₂). In some embodiments, in order for LF_{stochastic (x}₁, y₁, z₁) and LF_{stochastic (x}₂, y₂, z₂) to be uncorrelated, (x₁, y₁, z₁) and (x₂, y₂, z₂) should be separated by at least 0.01 mm or at least 0.05 mm or at least 0.1 or at least 0.2 mm or at least 0.3 mm or at least 0.5 mm or at least 1 mm. Thus, in some embodiment, the offset distance Off of FIGS. 2 and 10 should be at least 0.01 mm or at least 0.05 mm or at least 0.1 or at least 0.2 mm or at least 0.3 mm or at least 0.5 mm or at least 1 mm

In some embodiments, in order for LF_{reflected}_{_{—}}ₙₒₙ_{_{—}}_{brownian}(x₁, y₁, z₁)≈LF_{reflected}_{_{—}}ₙₒₙ_{_{—}}_{brownian(x}₂, y₂, z₂).

LF_{slowly}_{_{—}}_{fluctuating}(x₁, y₁, z₁)≈LF_{slowly}_{_{—}}_{fluctuating}(x₂, y₂, z₂), and LF_{regular}(x₁, y₁, z₁)≈LF_{regular}(x₂, y₂, z₂), then (x₁, y₁, z₁) and (x₂, y₂, z₂) should be separated by at most 10 cm or at most 5 cm or at most 3 cm or at most 2 cm or at most 1 cm or at most 0.5 mm or at most 0.25 or at most 1 mm. Thus, in some embodiments, the offset distance Off of FIG. 8 should be at most 10 cm or at most 5 cm or at most 3 cm or at most 2 cm or at most 1 cm or at most 0.5 mm or at most 0.25 or at most 1 mm

In this case, if A(t)=A₁(t)−A₂(t) represents LF(x₁, y₁, z₁)−LF(x₂, y₂, z₂), then it is possible to write, using equation (3), that A(t) represents

$$⌊ {LF}_{slowl y \_ fluctuating} ( {x}_{1} , {y}_{1} , {z}_{1} ) - {LF}_{slowl y \_ fluctuating} ( {x}_{2} , {y}_{2} , {z}_{2} ) ⌋ + \underbrace{\{ [ \begin{matrix}{LF}_{regular} ( {x}_{1} , {y}_{1} , {z}_{1} ) - \\ {LF}_{regular} ( {x}_{2} , {y}_{2} , {z}_{2} )\end{matrix} ] + [ \begin{matrix}{LF}_{stochastic} ( {x}_{1} , {y}_{1} , {z}_{1} ) - \\ {LF}_{stochastic} ( {x}_{2} , {y}_{2} , {z}_{2} )\end{matrix} ] \}}_{rapidly - fluctuating} . \qquad Eq ( 4 )$$

In the special case where (i) exact equality prevails—i.e. LF_{slowly}_{_{—}}_{fluctuating}(x₁, y₁, z₁)=LF_{slowly}_{_{—}}_{fluctuating}(x₂, y₂, z₂),

LF_{regular}(x₁, y₁, z₁)=LF_{regular}(x₂, y₂, z₂) and where (ii) rapidly fluctuating LF_{stochastic}(x₁, y₁, z₂) and LF_{stochastic}(x₂, y₂, z₂) are not correlated with each other, then A(t) is a completely stochastic signal (i.e. indicative of a time-varying speckles pattern or DLS measurement produced by scattering from the Brownian particles), in contrast to A₁(t) and A₂(t) where the stochastic components of the signal may only be some fraction less than ½, for example, less than 0.1 or less than 0.01. Practically, A(t) may also include some non-stochastic component. Nevertheless, in typical cases, the relative contribution of the non-stochastic component(s) (i.e., not due to scattering light on Brownian particles to generate a speckles pattern having a rapidly-varying intensity) to analog electric signal A(t) is less than the contribution of respective non-stochastic components to A₁(t) or A₂(t).

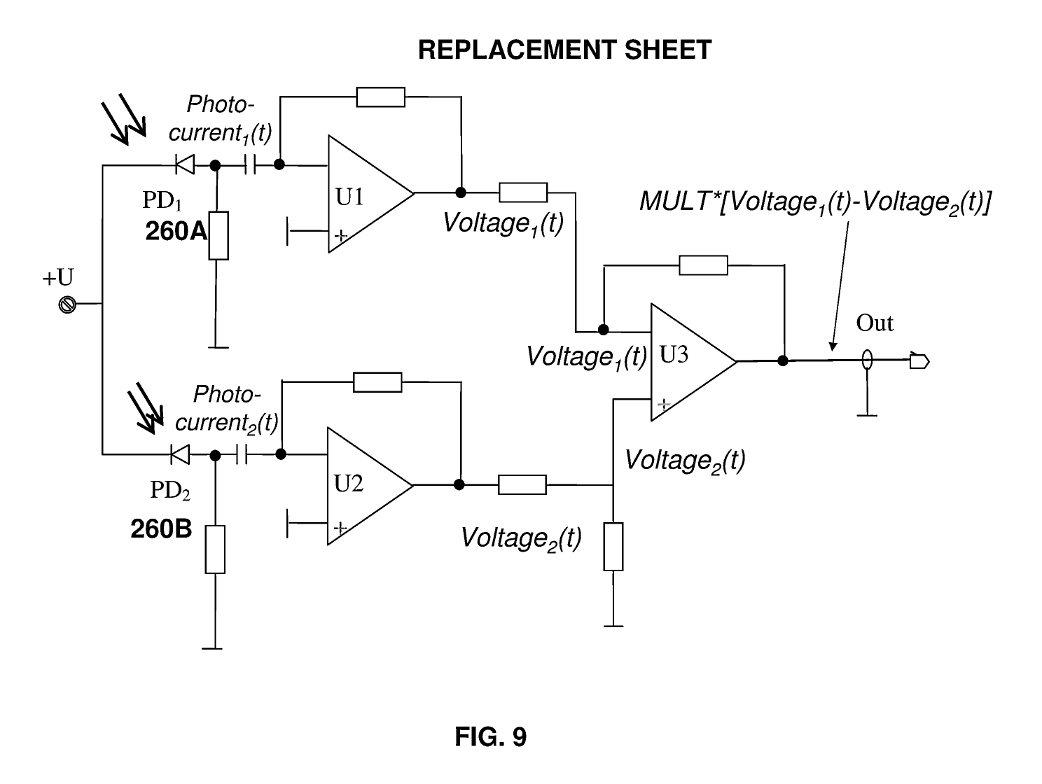

FIG. 9 is a circuit diagram of an exemplary analog electronic assembly 270 in accordance with some embodiments. Photocurrent Photocurrent₁(t)—generated by the first photodetector 260A is converted by a “first cascade” operational amplifier U1 from a “current analog signal” Photocurrentᵢ(t) to a “voltage analog signal” voltage₁(t)—Photocurrent₁(t) and voltage₁(t) are non-limiting examples of “first analog signals” A₁(t.

Photocurrent Photocurrent₂(t)—generated by the second photodetector 260B is converted by a “first cascade” operational amplifier U2 from a “current analog signal” Photocurrent₂(t) to a “voltage analog signal” voltage₂(t)—Photocurrent₂(t) and voltage₂(t) are non-limiting examples of “second analog signals” A₂(t).

“Second cascade” operational amplified U3 (i) receives as an input voltage₁(t) and voltage₂(t), and outputs a signal that is MULT[voltage₁(t)−voltage₂(t)], which is the difference between voltage₁(t) and voltage₂(t) multiplied by a constant whose value is MULT. It is noted that MULT[voltage₁(t)−voltage₂(t)] is one example of A(t.

The assembly of FIG. 9 is one example of a device that can generate a difference analog signal and/or a ‘hybrid’ analog signal.

FIGS. 10-11 illustrate the detector-generated electrical signal 930 or ‘analog substraction-derivatives’ thereof.

FIGS. 12A-12B illustrate identifying signal form parameters of a cardiac cycle. This may be performed in any manner—optically or otherwise, using DLS signals or any other signals—e.g. an additional detector may be employed, or the same detector may be ‘re-used.’

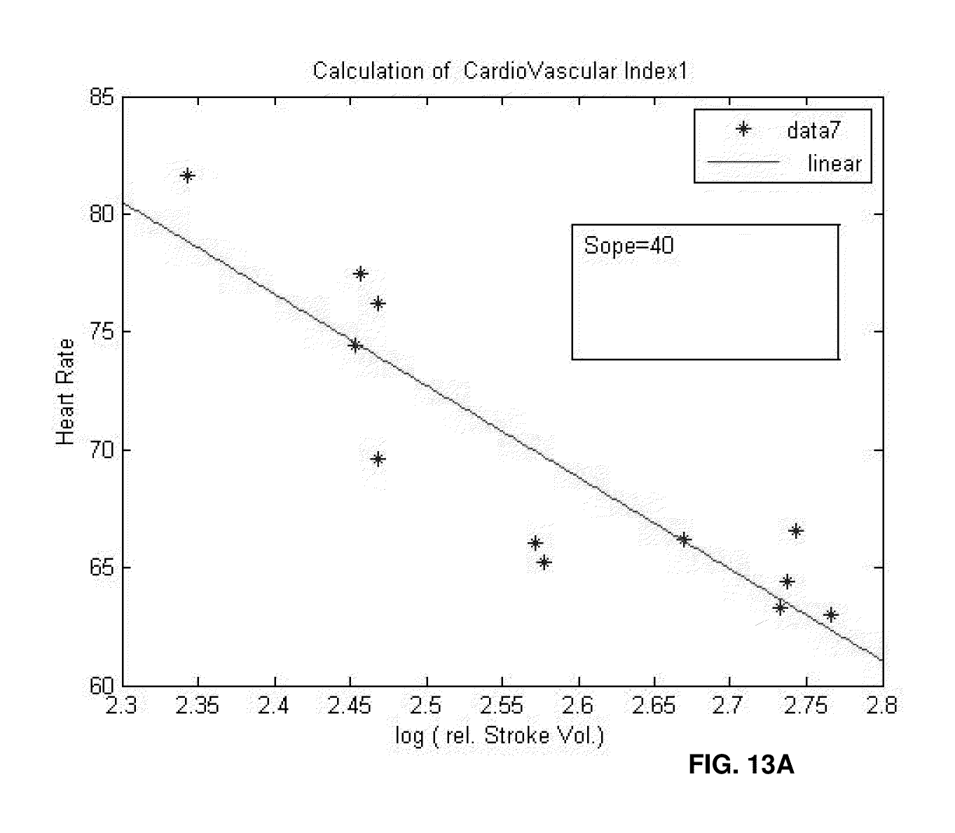

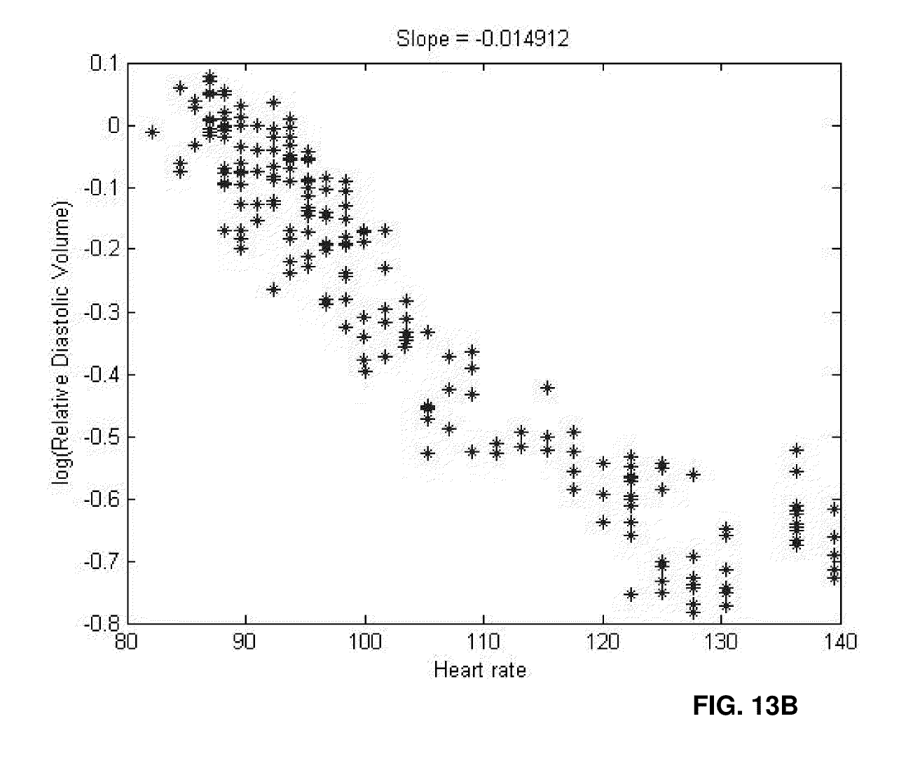

FIG. 13A-13B illustrate computing a corretlation between the multi-cycle stroke volume parameter data set 980 (i.e. logs of these values are on the x-axis of FIG. 13A) and cycle-specific heart rate/pulse data set 990 (see the y-axis of FIG. 13A). Preferably this correlation is performed by correlating not the ‘raw value’ of the stroke volume parameter but a logarithm function thereof—this allows one to obtain a linear correlation by using, for example, a linear regression.

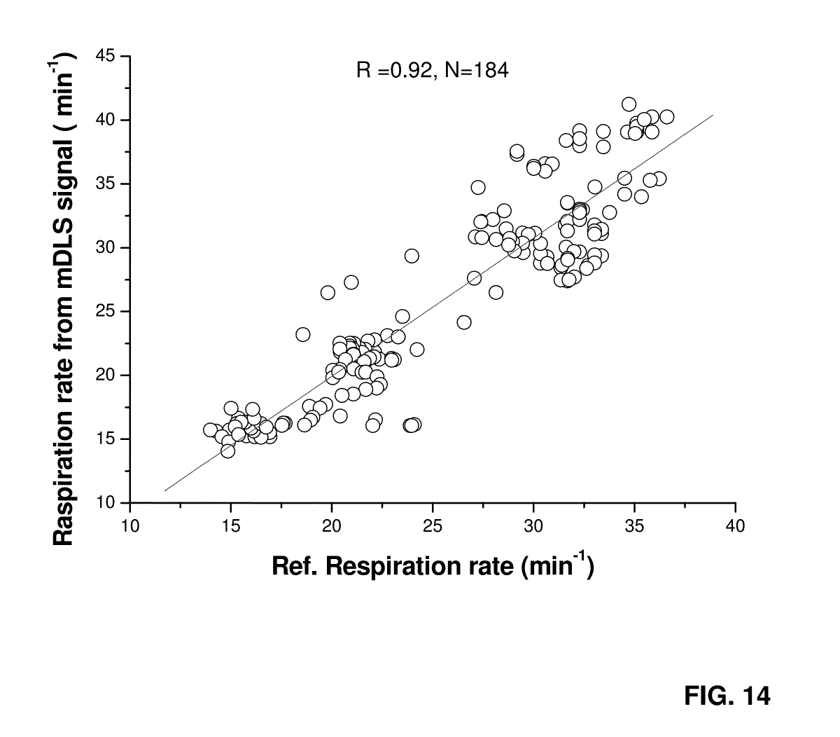

FIG. 14 relates to the technique of FIG. 6A.

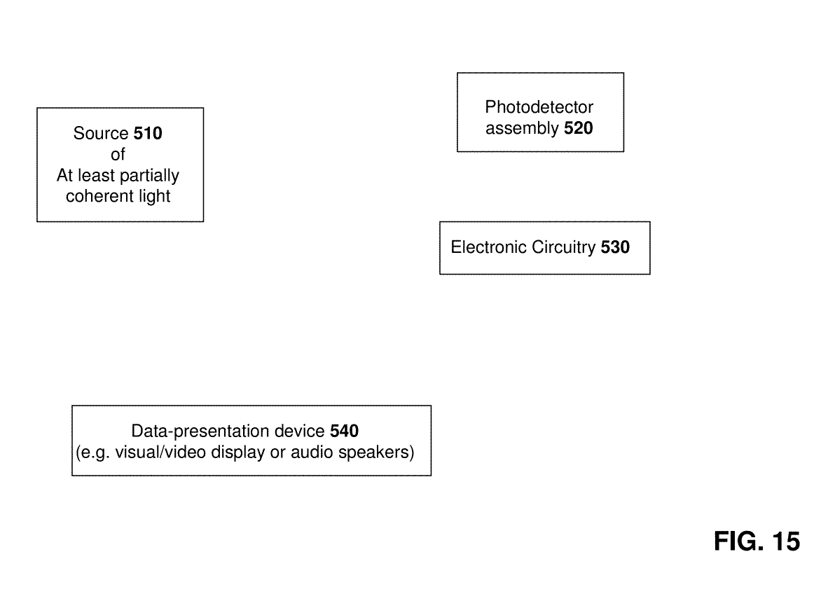

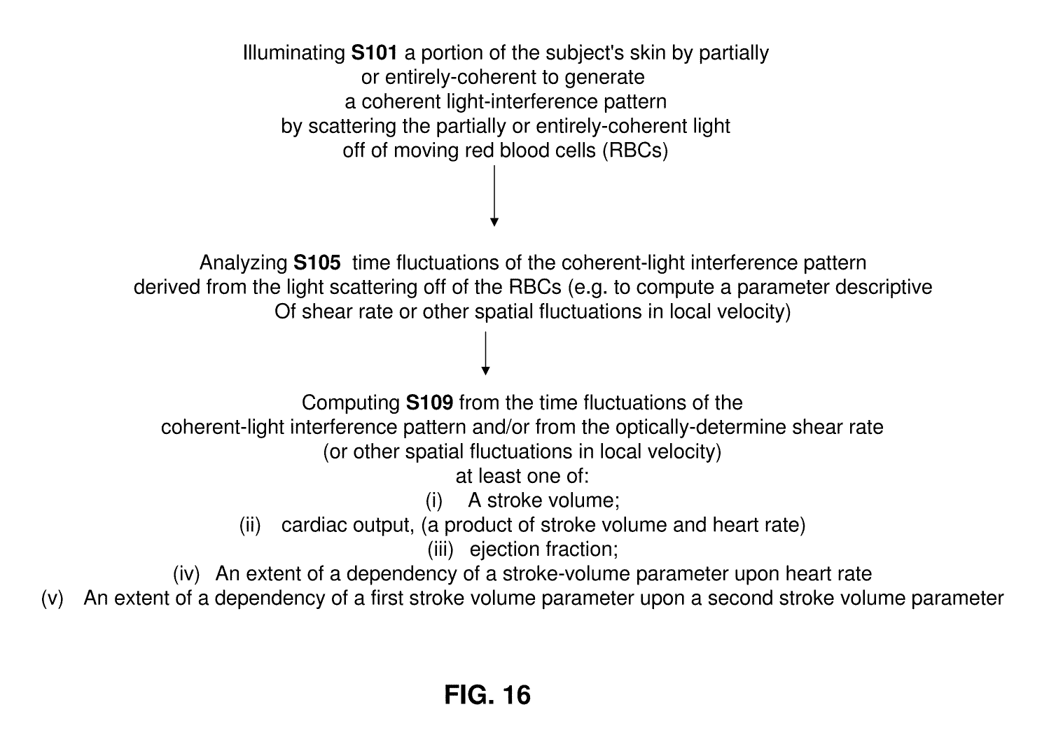

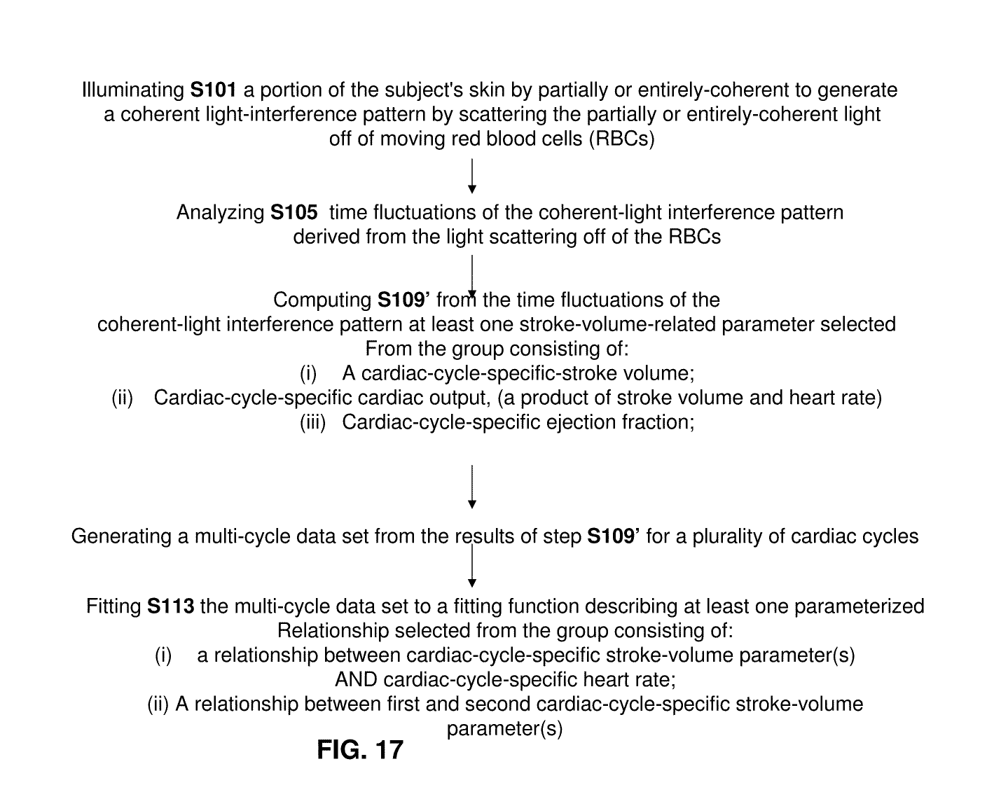

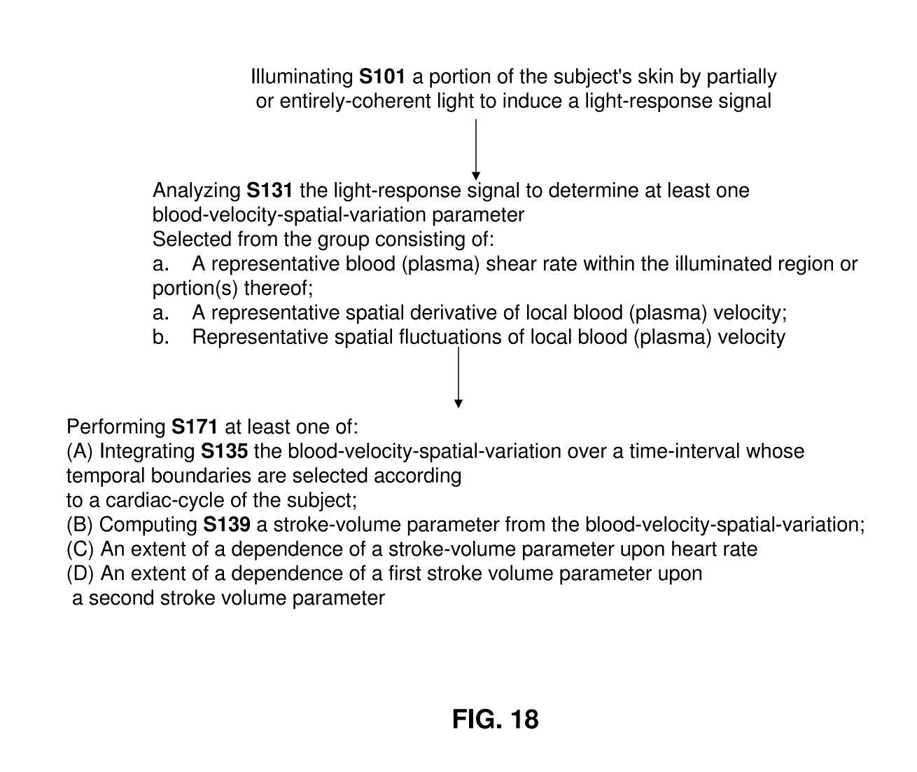

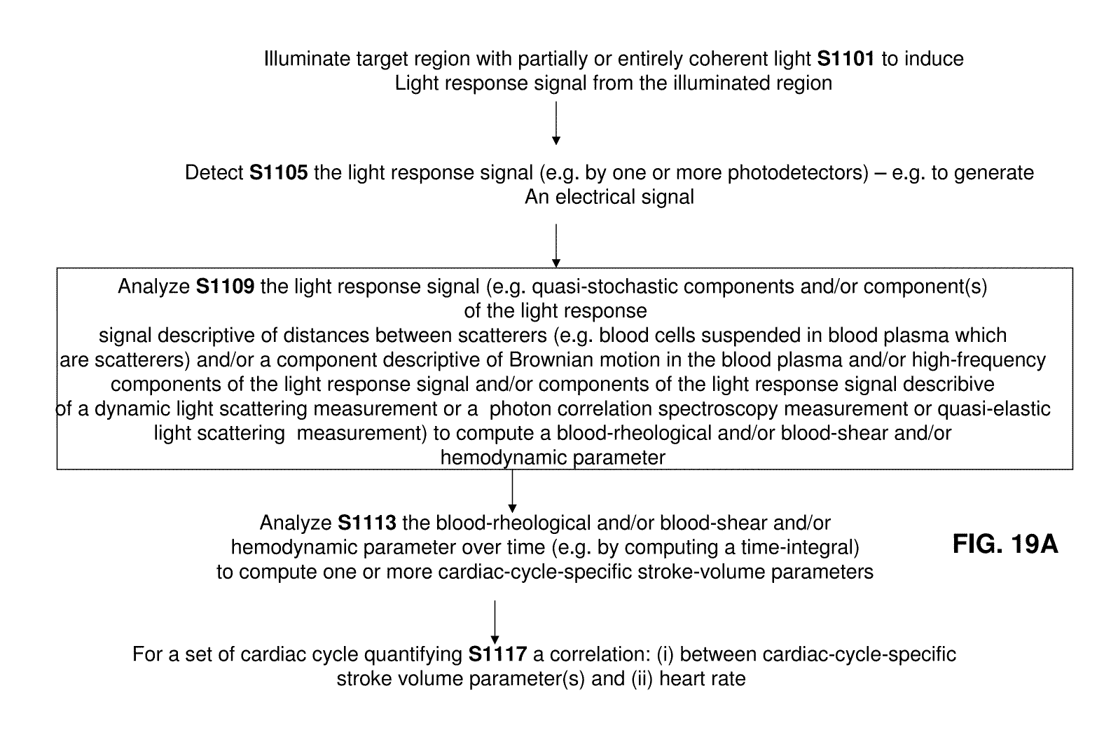

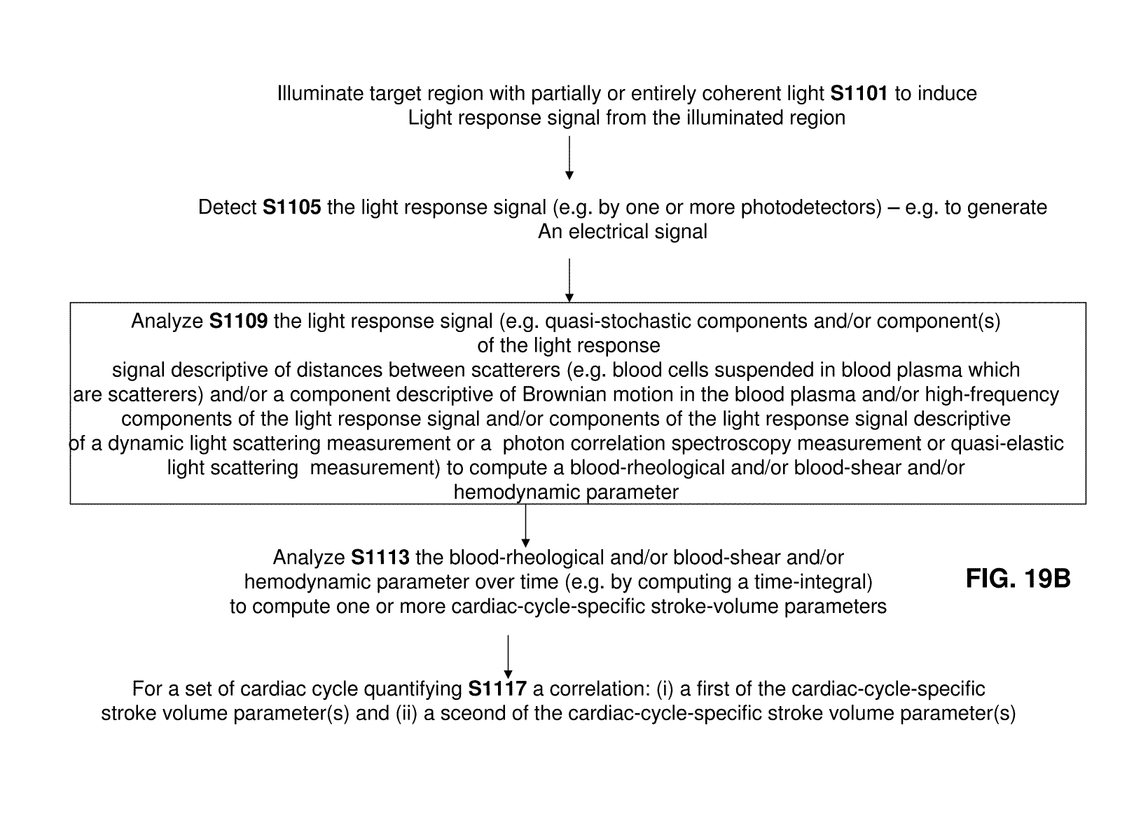

FIGS. 15-19 are taken from U.S. 61/884,202 and/or U.S. 61/884,975. The skilled artisan is directed to the text of these applications for an explanation.

Having thus described the foregoing exemplary embodiments it will be apparent to those skilled in the art that various equivalents, alterations, modifications, and improvements thereof are possible without departing from the scope and spirit of the claims as hereafter recited. In particular, different embodiments may include combinations of features other than those described herein. Accordingly, the claims are not limited to the foregoing discussion.

1. Apparatus for optically and non-invasively measuring a cardiovascular recovery metric of a subject, the apparatus comprising:
a. one or more sources of partially or entirely coherent light configured to illuminate a portion of the subject's skin to scatter partially or entirely coherent light off of moving red blood cells (RBCs) within the subject's blood to induce a location-dependent light field;
b. a plurality of photodetectors including first and second photodetectors respectively configured to detect light of the location-dependent light field at first and second locations to respectively generate first and second analog electrical signals that respectively describe the light field at the first and second locations;
c. analog circuitry configured to generate a difference analog electrical signal that describes a difference between the first and second analog signals; and
d. analysis electronic circuitry configured to compute the cardiovascular recovery metric from the difference analog electrical signal or a derivative thereof by:
i. deriving from the difference analog electrical signal or from the derivative thereof a blood-shear-parameter signal describing a blood-shear-parameter of the subject over a period of time;
ii. for each given cardiac cycle of a plurality of cardiac cycles, obtaining or computing respective cardiac-cycle signal-form characteristics thereof;
iii. generating a multi-cycle stroke-volume-parameter data set by respectively computing, for each given cardiac cycle of the plurality of cardiac cycles, a respective cardiac-cycle-specific stroke-volume parameter by subjecting the blood-shear-parameter signal to a temporal analysis in accordance with the respective cardiac-cycle signal-form characteristics specific for the given cardiac cycle;
iv. generating a multi-cycle pulse-rate data set by measuring, for each given cardiac cycle of the plurality of cardiac cycles, a respective pulse-rate specific for the given cardiac cycle;
v. quantifying a correlation between the multi-cycle stroke-volume parameter data set and the multi-cycle pulse rate data set; and
vi. computing the cardiovascular recovery metric of the subject from the quantified magnitude.

2. The apparatus of claim 1 configured to the correlation between the multi-cycle stroke-volume parameter data set and the multi-cycle pulse rate data set by computing logarithms of the values of the correlation between the multi-cycle stroke-volume parameter data set and the multi-cycle pulse rate data set.

3. The apparatus of claim 1 configured to the correlation between the multi-cycle stroke-volume parameter data set and the multi-cycle pulse rate data set by computing logarithms of the values of the correlation between the multi-cycle stroke-volume parameter data set and the multi-cycle pulse rate data set and then computing a strength of a linear correction between the values of the logarithms and the values of the per-cycle pulse values of the multi-cycle pulse rate data set.

4. Apparatus for optically and non-invasively measuring a respiratory rate of a subject or a function thereof, the apparatus comprising:
a. one or more sources of partially or entirely coherent light configured to illuminate a portion of the subject's skin to scatter partially or entirely coherent light off of moving red blood cells (RBCs) within the subject's blood to induce a location-dependent light field;
b. a plurality of photodetectors including first and second photodetectors respectively configured to detect light of the location-dependent light field at first and second locations to respectively generate first and second analog electrical signals that respectively describe the light field at the first and second locations;
c. analog circuitry configured to generate a difference analog electrical signal that describes a difference between the first and second analog signals; and
d. analysis electronic circuitry configured to compute the cardiovascular recovery metric from the difference analog electrical signal or a derivative thereof by:
i. deriving from the difference analog electrical signal or from the derivative thereof a blood-shear-parameter signal describing a blood-shear-parameter of the subject over a period of time;
ii. for each given cardiac cycle of a plurality of cardiac cycles, obtaining or computing respective cardiac-cycle signal-form characteristics thereof;
iii. generating a multi-cycle stroke-volume-parameter signal by respectively computing, for each given cardiac cycle of the plurality of cardiac cycles, a respective cardiac-cycle-specific stroke-volume parameter by subjecting the blood-shear-parameter signal to a temporal analysis in accordance with the respective cardiac-cycle signal-form characteristics specific for the given cardiac cycle;
iv. temporally analyzing the stroke-volume-parameter signal to characterize temporal fluctuations thereof; and
v. computing the respiratory rate from the characterized temporal fluctuations.

5. The apparatus of claim 4 wherein the respiratory rate is computed by (i) identifying or quantifying a dominant frequency of the per-cycle-stroke-volume signal; and (ii) deriving the respiratory rate from the dominant frequency.

6. The apparatus of claim 4 configured to the correlation between the multi-cycle stroke-volume parameter data set and the multi-cycle pulse rate data set by computing logarithms of the values of the correlation between the multi-cycle stroke-volume parameter data set and the multi-cycle pulse rate data set.

7. The apparatus of claim 4 configured to the correlation between the multi-cycle stroke-volume parameter data set and the multi-cycle pulse rate data set by computing logarithms of the values of the correlation between the multi-cycle stroke-volume parameter data set and the multi-cycle pulse rate data set and then computing a strength of a linear correction between the values of the logarithms and the values of the per-cycle pulse values of the multi-cycle pulse rate data

8. A method of detecting apnea comprising:
a. monitoring a respiratory rate using the apparatus of claim 4; and
b. if the respiratory rate falls below a threshold, generating an alert signal.

9. A method of detecting apnea comprising:
a. monitoring a respiratory rate using the apparatus of claim 4;
b. receiving the data of the monitoring of the respiratory rate by an apnea-event classifier; and
c. in accordance with output of the apnea-event classified, generating an alert signal.

10. The method of claim 8 wherein the alert signal is a visual alert signal or an audio alert signal.
